(19) United States
(12) Patent Application Publication
(10) Pub. No.: US 2004/0039432 A1
(43) Pub. Date: Feb. 26, 2004

(54) Topical therapeutic thermos for humans and other animals

(76) Inventor: Gerald E. Warriner, West Melbourne, FL (US)

Correspondence Address: GERALD E. Warriner, 9037-H York Lane, West Melbourne, FL, 32904, US

(21) Appl. No.: 10/226,868

(22) Filed: Aug. 24, 2002

(51) Int. Cl. A61F 7/00

(52) U.S. Cl. 607/108; 607/111

(57) ABSTRACT

The Topical Therapeutic Thermos for Humans and Other Animals herein described has been invented for the purpose of providing; an effective means of reducing and/or preventing swelling and pain that is often associated with physical injury, strenuous work and/or exercise; an effective means of reducing swelling and pain following surgery; an effective means of supplying cooling temperature uniformly to areas of a living body in order to avoid injury caused by swelling due to repetitive physical stress; an effective means of supplying warming temperature uniformly to areas of a living body to aid in physical therapy and speed in recovery of injury; and an effective means of extending the athletic lives of professional and amateur human and non-human athletes.

The Topical Therapeutic Thermos for Humans and Other Animals contains an outer casing, an inflatable bladder, a multi-cellular bladder containing a liquid, a gel, and/or solids that may be repeatedly and/or alternatively heated or cooled, a cloth liner, an inflation pump, a pressure release valve, a temperature indicator and a zipper or other closure device.

### CROSS-REFERENCE TO RELATED APPLICATIONS

Not applicable.

### STATEMENT REGARDING FEDERALLY SPONSORED RESEARCH OR DEVELVPMENT

Not Applicable

### REFERENCE TO SEQUENCE LIST, A TABLE, OR A COMPUTER PROGRAM LISTING COMPACT DISC APPENDIX

Not Applicable

### BACKGROUND OF THE INVENTION

1. Current Art

The Current Art invention is a Topical Therapeutic Thermos for humans and other animals that will completely or partially enclose specific body parts (such as a head, face, neck, shoulder, bicep, elbow, wrist, hip, thigh, knee, calf, ankle or foot) and that will consist of a combination of an outer casing that is contiguous with an insulation membrane that is contiguous with an inflatable bladder that is contiguous with a multi-cellular bladder that contains a gel, a liquid or solids that may be repeatedly and alternatively heated or cooled that is contiguous with a cloth separator that will be in contact with a body part when the thermos is in use and that also contains an air pump and air-pressure valve, a chemical or mechanical temperature measuring device with a visual display and a zipper or other means of opening and closing the thermos.

2. Prior Art

An exterior casing that completely or partially encloses a portion of human body part or the body part of another animal such as a head, face, neck, shoulder, bicep, elbow, wrist, hip, thigh, knee, calf, ankle or foot is not patentable in and of its self because exterior casings have been easily obtainable and applicable for such common use for decades as exampled by U.S. Pat. No. 1,257,086 that relates to an exterior casing, current U.S. Class: 36/2.6; 36/88; 36/141, Hart W. Marcellus, February 1918, expired.

An inflatable bladder utilized to apply pressure against a human body part or the body part of another animal such as a head, face, neck, shoulder, bicep, elbow, wrist, hip, thigh, knee, calf, ankle or foot is not patentable in and of its self because inflatable bladders have been easily obtainable and applicable for such common use as exampled by U.S. Pat. No. 3,701,349 that relates to an inflatable bladder, current U.S, Class 128/89, Leighton W. Larson, October, 1972, expired.

Single cell and/or multi-cell devices containing liquid, gel or solids that may be repeatedly and/or alternatively heated or cooled and placed against a human body part or the body part of another animal are not patentable in and of themselves as exampled by U.S. Pat. No. 2,602,302 that relates to such multi-cell devices, current U.S. Class 62/530; 607/112; D24/206, Noel J. Poux, July 1952, expired.

A cloth liner between a hot or cold substance and the skin of human body part or the body part of another animal is not patentable in and of its self because a cloth fabric is and has been easily obtainable and applicable for such common use as exampled by U.S. Pat. No. 3,945,047, that relates to such a cloth liner, current U.S. Class 2/24, Richard P. Jarrell, Jr., March 1976, expired.

A zipper is not patentable in and of its self because it is and has been easily obtainable and applicable for such common use for decades as exampled by U.S. Pat. No. 3,930,667, current U.S. Class 280/730.1; 2/DIG3; 280/290; 280/729; 280/733; 280/734; 441/103, Andrew J. Osuchowski, January 1976, expired.

Temperature measuring devices such as a chemical and/or mechanical thermometers are not in and of themselves patentable as they have been easily obtainable and applicable for common use as exampled by U.S. Pat. No. 1,942,516, current U.S. Class 136/224, 136,230, 374/182, Bradford Noyes, Jr., January 1934, expired.

Inflation pumps are not in and of themselves patentable as they have been easily obtainable and applicable for common use as exampled by U.S. Pat. No. 2,006,878, current U.S. Class 137/614.11, 251/126, 600/498, Erich Bandoly, July 1935, expired.

Methods of heating required for the use of the current invention related to this application such as conventional electrical ovens and microwave ovens have been easily obtainable and applicable for common use as exampled by U.S. Pat. No. 3,943,319 current U.S. Class 219/739, 126/190, 219/744, 219/756, Hirai; Kazumi et al., March 1976, expired and U.S. Pat. No. 3,935,415, current U.S. Class 219/750, 219/729; 219/762, Donald G. Moore, January 1976, expired.

Methods of cooling required for the use of the current invention related to this application such as electrical refrigerators and freezers have been easily obtainable and applicable for common use as exampled by U.S. Pat. No. 3,619,482, U.S. Class 312/406, 52/631, 62/DIG13, 220/592.09, 312/236, Charles W. Haag, January 1976, expired and U.S. Pat. No. 3,935,714, current U.S. Class 62/187, 62/265, 62/267, Samuel J. Woolley, February 1976, expired.

Since all of the components included in the current invention are not in and of themselves patentable, they will not be individually claimed, but they will be claimed as being components of the invention and the patent must be based on the relationships of the components to each other and to the functions, uses and benefits that the components and relationships of the invention provide.

Prior art does not include any patent that contains all of the components utilized by the current art invention. Furthermore, no patented invention, current or expired, refers to a topical therapeutic thermos, however, there are a number of patents that warrant inspection.

The current art has application for other animals as well as humans and in all cases may be modified for use following surgery on humans and other animals.

Prior Art, Humans

U.S. Pat. Nos. 6,322,530-5,868,690-5,733,321-5,717,996 5,514,081-5,435,009-5,363,632-5,000,164 4,805,601-4,671,267-4,403,265-3,701,349

Prior Art, Equine

U.S. Pat. No. 5,363,632

Special Note: U.S. Pat. No. 5,868,690, U.S. current U.S. Class 601/151; 128/DIG20; 601/15; 601/152; 602113; 602/14, Clement G. Eischen, Sr., February 1999 ‘appears’ to be similar to the current art invention submitted herein, however, upon close inspection it may be shown that the prior art contains two inflation bladders with two connecting passageways, a pocket to hold a “thermal treatment device” plus a “platform-defining sling within the bladder”, none of which are contained in the current art invention.

Additionally, the prior art invention does not include;

A flexible multi-cell bladder that contains a liquid, gel or solid that may be repeatedly and/or alternatively heated or cooled and placed against a portion of a human or other animal body part for therapeutic purposes;

A cloth liner to protect the skin from direct contact with the source of heat and cold;

A zipper;

A temperature measuring device; or * A pump and pressure valve to inflate the ‘inflatable boot’.

In summary, the prior art has five or six components and features that the current art does not contain and the current art has five or six components and features that the prior art does not contain.

### BRIEF SUMMARY OF THE INVENTION

The Topical Therapeutic Thermos for Humans and Other Animals herein described has been invented for the purpose of providing;

an effective means of reducing and/or preventing swelling and pain that is often associated with physical injury, strenuous work and/or exercise;

an effective means of reducing swelling and pain following surgery;

an effective means of supplying cooling temperature uniformly to areas of a living body in order to avoid injury caused by swelling due to repetitive physical stress;

an effective means of supplying warming temperature uniformly to areas of a living body to aid in physical therapy and speed recovery of injury; and

an effective means of extending the athletic lives of professional and amateur human and non-human athletes.

The Topical Therapeutic Thermos for Humans and Other Animals contains an outer casing, an inflatable bladder, an insulation membrane, a multi-cellular bladder containing a liquid, a gel, and/or solids that may be repeatedly and/or alternatively heated or cooled, a cloth liner, an inflation pump, a pressure release valve, a temperature indicator and a zipper or other closure device.

### BRIEF DESCRIPTION OF THE DRAWINGS

Seven drawings, FIG. 1 through FIG. 7 are included in these specifications.

FIG. 1 is a vertical section of a Topical Therapeutic Knee Thermos for humans.

FIG. 2 is a vertical section of a Topical Therapeutic Elbow Thermos for humans.

FIG. 3 is a vertical section of a Topical Therapeutic Wrist Thermos for humans.

FIG. 4 is a vertical section of a Topical Therapeutic Ankle Thermos for humans.

FIG. 5 is a side elevation of a Topical Therapeutic Ankle Thermos for humans.

FIG. 6 is a horizontal section of a Topical Therapeutic Ankle Thermos for humans.

FIG. 7 is a vertical section of a Topical Therapeutic Lower Leg Thermos for horses.

The Topical Therapeutic Thermos for Humans and Other Animals contains an outer casing 1, an inflatable bladder 2, a multi-cellular bladder containing a liquid, a gel, and/or solids that may be repeatedly and/or alternatively heated or cooled 3, a cloth liner 4, an inflation pump and pressure release valve 5, a top, bottom and/or end binding 6, a temperature indicator 7, and a zipper or other closure device 8.

### DETAILED DESCRIPTION OF THE INVENTION

The Topical Therapeutic Thermos for Humans and other Animals invention allows the user to uniformly apply hot or cold temperature to specific areas of the body such as the head, face, neck, shoulders, elbows, wrists, hips, thighs, knees, calves, ankles and feet.

Each area of the body, such as those identified above, requires a differently shaped thermos that will follow the general form of the body part that is to receive the heating or cooling effect desired.

Uniformity in the application of hot and cold temperatures is achieved by; one, completely surrounding the body part (when possible) with the thermos; two, applying uniform pressure to the area; and three, providing insulation to maintain the desired temperature as long as needed to safely achieve the desired result.

Topical Therapeutic Thermos Construction

The thermos is constructed utilizing; one, an exterior casing made of semi-rigid plastic such as Kydex or a flexible plastic or cloth fabric; two, an inflatable bladder made of flexible rubber or plastic; and three, a multi-cellular bladder made from flexible plastic film containing a liquid, gel or solids that may be repeatedly and/or alternatively heated or cooled. A cloth membrane or similar component will be positioned between the multi-celled bladder and that portion of the body being cooled or heated and an insulation membrane will be positioned between the inflatable bladder and the multi-cellular bladder.

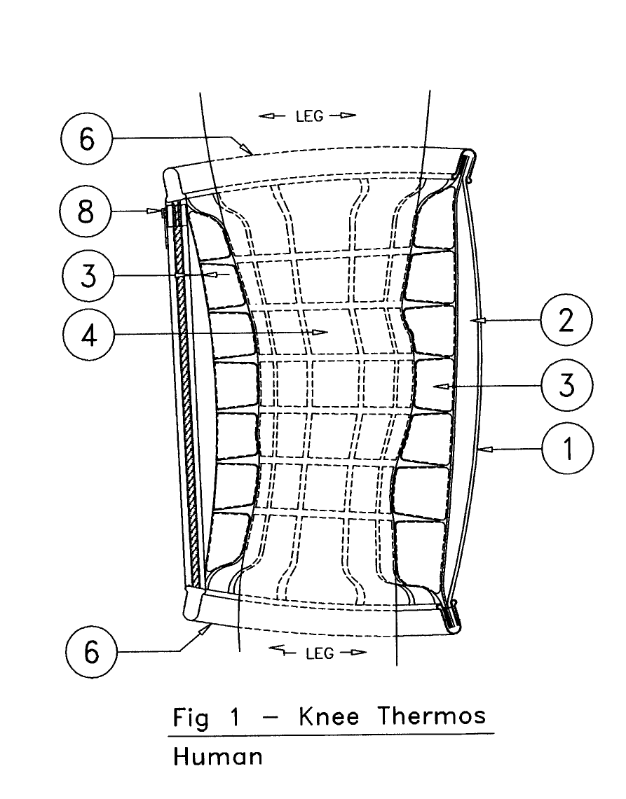

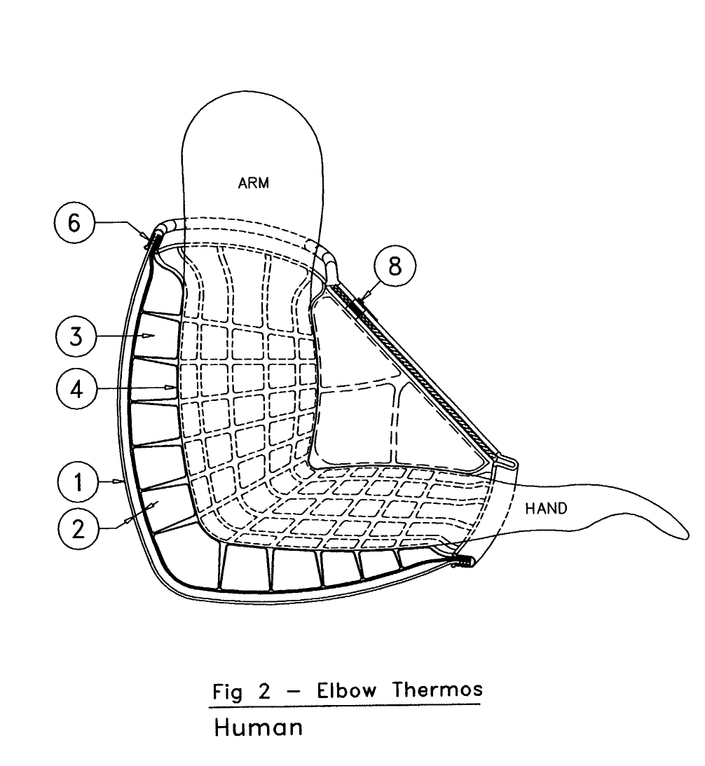

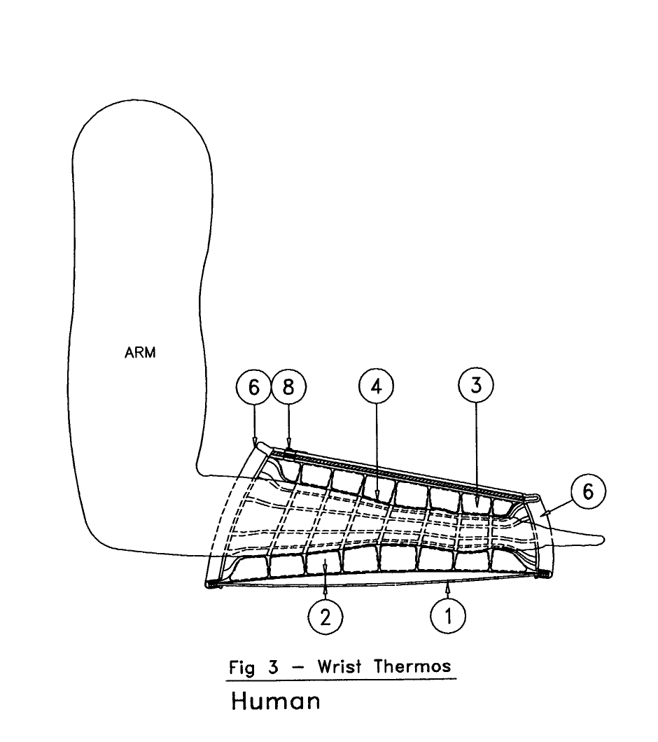

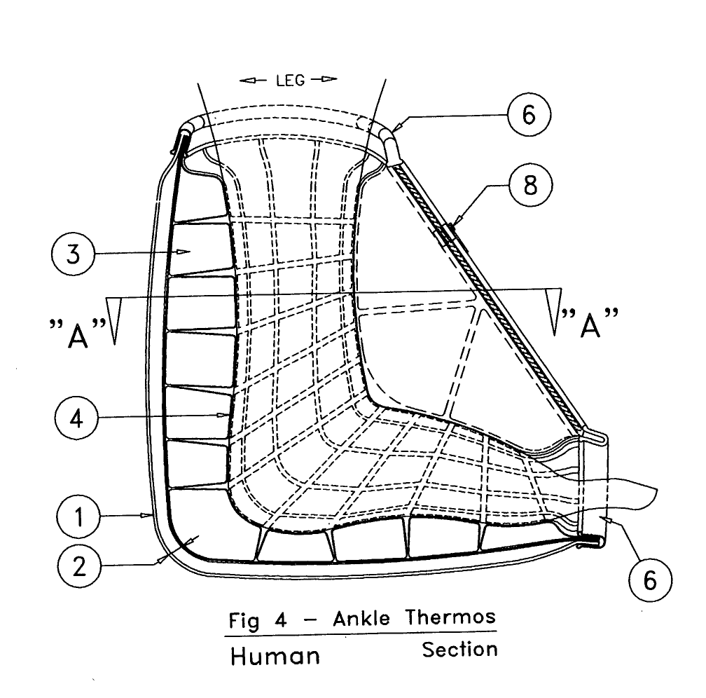

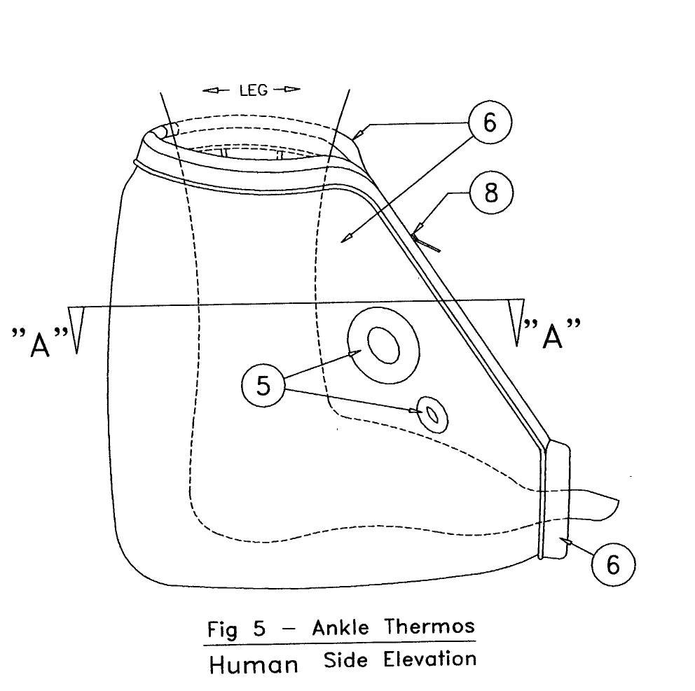

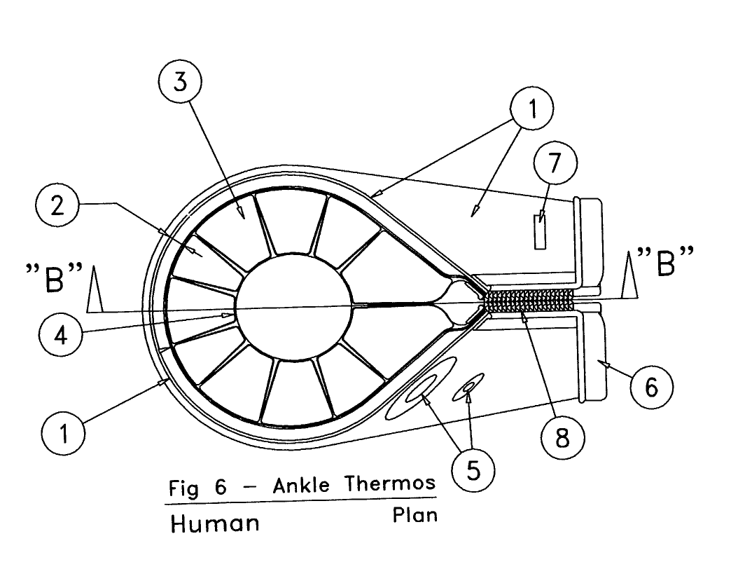

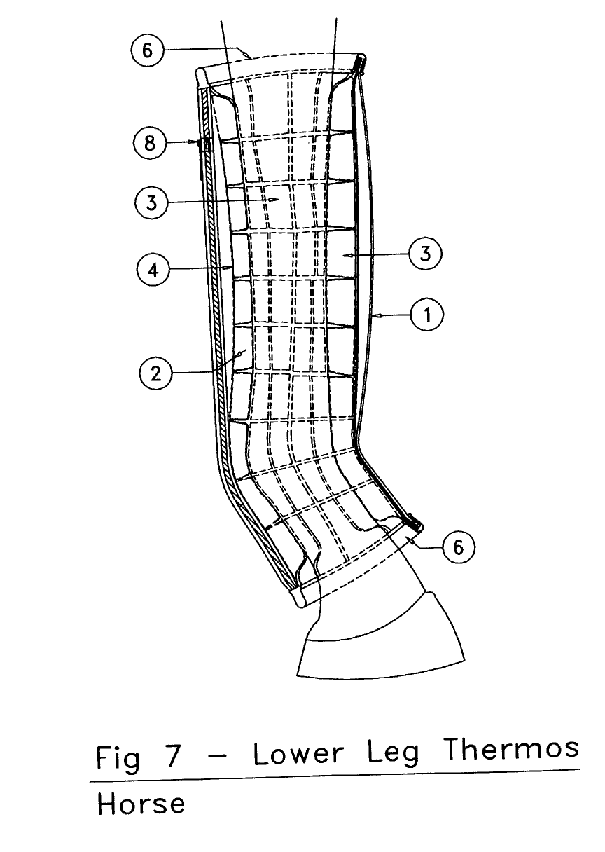

Each bladder identified above will be sealed along their perimeters and will also be attached along their perimeters to each other and to the cloth separator and insulation membrane that are also identified above. The perimeters occur along the binders located at openings at the tops, bottoms and/or ends of the thermoses and along each side of the zippers shown in drawings FIG. 1 through FIG. 7.

The exterior casing, internal bladders, insulation membrane and cloth separator are all contoured to accommodate the general shape of the body part for which the thermos is configured and all internal components will ‘nest’ within the casing in close and uniform contact.

The individual cells that make up the multi-cellular bladder will each be contoured to allow for expansion and deformation required to accommodate the contours of the body part with which they come into contact. The innermost membrane of the multi-cellular bladder is embossed or ‘drawn’ into a waffle-like pattern of cells. The outermost membrane of the multi-cellular bladder is not embossed.

The exterior casing is penetrated by a manually operated air-pressure pump and air-pressure relief valve. The inflatable bladder is positioned between the exterior casing and the multi-cellular bladder. The pump produces air-pressure within the inflatable bladder and thereby presses the cellular bladder against the surface of the body part.

A chemical or mechanical temperature device also penetrates the exterior casing for the purpose of informing the user of the current temperature within the thermos.

The air space within the inflatable bladder provides additional insulation between the cellular bladder and the exterior casing.

An opener/closure device shown as a zipper in FIG. 1 through FIG. 7 is provided to easily put on or take off the thermos. Such a device could be a zipper, laces, straps, hooks or overlapping Velcro fasteners.

Trim and/or cosmetic cover may enclose the casing and may be fabric, rubber, cloth, plastic and/or leather.

The thermos may be cooled to the desired temperature range by placing it in a refrigerator, freezer or similar device or the thermos may be heated to the desired temperature range by placing it in a conventional oven, microwave oven or similar device.

I claim as my invention:

1. A Topical Therapeutic Thermos for Humans & Other Animals that completely or partially enclose specific body parts (such as a head, face, neck, shoulder, bicep, elbow, wrist, hip, thigh, knee, calf, ankle or foot) and that consists of a combination of an outer casing that is contiguous with an insulation membrane that is contiguous with an inflatable bladder that is contiguous with a cloth separator that will be in contact with a body part when the thermos is in use.

2. A Topical Therapeutic Thermos for Humans & Other Animals as claimed in claim 1 with an exterior casing made of semi-rigid plastic such as Kydex or a flexible plastic film or fabric with high tinsel strength.

3. A Topical Therapeutic Thermos for Humans & Other Animals as claimed in claim 1 with an inflatable bladder made of flexible rubber or plastic.

4. A Topical Therapeutic Thermos for Humans & Other Animals as claimed in claim 1 with an insulation membrane with or without a reflective surface positioned between the inflatable bladder and the multi-cellular bladder.

5. A Topical Therapeutic Thermos for Humans & Other Animals as claimed in claim 1 with a multi-cellular bladder made from flexible plastic film containing a liquid, gel or solids that may be repeatedly and/or alternatively heated or cooled.

6. A Topical Therapeutic Thermos for Humans & Other Animals as claimed in claim 1 with a cloth membrane or similar component positioned between the multi-celled bladder and that portion of the body being cooled or heated.

7. A Topical Therapeutic Thermos for Humans & Other Animals as claimed in claim 1 with each bladder being sealed along their perimeters and attached along their perimeters to each other and to the cloth separator and insulation membrane and with the perimeters occuring along the binders located at openings at the tops, bottoms and/or ends of the thermoses and along each side of the zippers shown in drawings FIG. 1 through FIG. 7.

8. A Topical Therapeutic Thermos for Humans & Other Animals as claimed in claim 1 with the exterior casing, internal bladders, insulation membrane and cloth separator all contoured to accommodate the general shape of the body part for which the thermos is configured and with all internal components ‘nested’ within the casing in close and uniform contact.

9. A Topical Therapeutic Thermos for Humans & Other Animals as claimed in claim 1 with the individual cells that make up the multi-cellular bladder each contoured to allow for expansion and deformation required to accommodate the contours of the body part with which they come into contact and with the innermost membrane of the multi-cellular bladder embossed or ‘drawn’ into a waffle-like pattern of cells and with the outermost membrane of the multi-cellular bladder not being embossed and with the embossed membrane and the un-embossed membrane being fused or adhesively joined together to form individual cells.

10. A Topical Therapeutic Thermos for Humans & Other Animals as claimed in claim 1 with the exterior casing penetrated by a manually operated air-pressure pump and air-pressure relief valve and with the inflatable bladder positioned between the exterior casing and the multi-cellular bladder for the purpose of producing air pressure within the inflatable bladder and thereby pressing the cellular bladder against the surface of the body part when the bladder is inflated.

11. A Topical Therapeutic Thermos for Humans & Other Animals as claimed in claim 1 with a chemical or mechanical temperature device that penetrates the exterior casing for the purpose of informing the user of the current temperature within the thermos.

12. A Topical Therapeutic Thermos for Humans & Other Animals as claimed in claim 1 with an opener/closure device shown as a zipper in FIG. 1 through FIG. 7 provided for the purpose of putting on or taking off the thermos or with an opener/closure device such as laces, straps, hooks or overlapping Velcro type fasteners.

13. A Topical Therapeutic Thermos for Humans & Other Animals as claimed in claim 1 with trim and/or cosmetic cover made of fabric, rubber, cloth, plastic and/or leather enclosing the casing for appearance and/or for marketing considerations

14. A Topical Therapeutic Thermos for Humans & Other Animals as claimed in claim 1 with the thermos being able to be cooled to the desired temperature range by placing it in a refrigerator, freezer or similar device or with the thermos being able to be heated to the desired temperature range by placing it in a conventional oven, microwave oven or similar device.
